(19) United States
(12) Patent Application Publication
(10) Pub. No.: US 2014/0358116 A1
(43) Pub. Date: Dec. 4, 2014

(54) METHODS FOR DELIVERING AN ANTI-CANCER AGENT TO A TUMOR

(71) Applicant: UNIVERSITY OF UTAH RESEARCH FOUNDATION, Salt Lake City, UT (US)

(72) Inventors: Hamid GHANDEHARI, Salt Lake City, UT (US); Adam GORMLEY, London (GB); Abhijit RAY, Salt Lake City, UT (US); Nate LARSON, Murray, UT (US)

(73) Assignee: UNIVERSITY OF UTAH RESEARCH FOUNDATION, Salt Lake City, UT (US)

(21) Appl. No.: 14/461,888

(22) Filed: Aug. 18, 2014

Related U.S. Application Data
(62) Division of application No. 13/809,595, filed on Mar. 28, 2013.
(60) Provisional application No. 61/363,875, filed on Jul. 13, 2010.

(51) Int. Cl. A61K 41/00; A61N 5/06

(52) U.S. Cl. CPC A61K 41/0052; A61N 5/062
USPC 604/501

(57) ABSTRACT

Described herein are methods for delivering an anti-cancer agent to a tumor in a subject. The method involves
administering to the subject (i) gold particles and (ii) at least one-anti-cancer agent directly or indirectly bonded to the macromolecule and/or unbound to the macromolecule; and
exposing the tumor to light for a sufficient time and wavelength in order for the gold particles to achieve surface plasmon resonance and heating the tumor.

### CROSS REFERENCE TO RELATED APPLICATION

This application is a divisional application of U.S. application Ser. No. 13/809,595, filed on Mar. 28, 2013, which is a U.S. national phase application under 35 USC 371 of international application number PCT/US2011/043808, filed Jul. 13, 2011, which claims priority to upon U.S. Provisional Application Ser. No. 61/363,875, filed Jul. 13, 2010. These applications are hereby incorporated by reference in their entirety for all of their teachings.

### ACKNOWLEDGEMENTS

The research leading to this invention was funded in part by the National Institutes of Health, Grant Nos. R01 EB007171 and R01 DE019050-01, the National Science Foundation, Grant No. ID 0835342, and a Department of Defense Prostate Cancer Predoctoral Training Award (PC094496). The U.S. Government has certain rights in this invention.

### BACKGROUND

Gold particles have been investigated to treat cancer by photothermal therapy. Local heat generated by high energy laser excitation of their surface plasmons has the capacity to kill malignancies by photothermal lysis of nearby cancerous cells. Unfortunately, limited tissue penetration depths of light may ultimately limit the clinical applicability of this technology. Current strategies for photothermal therapy utilize passive diffusion of their nanoconstructs for delivery to the tumor. Low intratumoral concentrations and large plasma membrane separation distances of nanoconstructs may result thereby limiting the lethality at low laser energies. Therefore, it is desirable that photothermal strategies be developed to maximize efficacy with minimal light energy.

### SUMMARY

Described herein are methods for delivering an anti-cancer agent to a tumor in a subject. The method involves

administering to the subject (i) gold particles and (ii) at least one-anti-cancer agent directly or indirectly bonded to the macromolecule and/or unbound to the macromolecule; and

exposing the tumor to light for a sufficient time and wavelength in order for the gold particles to achieve surface plasmon resonance and heating the tumor.

The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows an exemplary synthetic procedure for making a modified gold particle with a targeting group.

FIG. 2 shows (A) light absorption profile and (B) transmission electron micrograph of GNRs. Panel A shows the absorbance profile of CTAB stabilized GNRs (GNRs), CTAB stabilized GNRs with 3.5% NaCl (GNRs+NaCl), as well as RGDfK-PEG-GNRs with and without 3.5% NaCl (RGDfK-GNRs±NaCl). Without the polymer coating GNRs aggregate in the presence of NaCl whereas those stabilized with PEG-RGDfK are stable in the presence of salt.

FIG. 3 shows GNR binding and uptake by (A) high-resolution dark field microscopy and (B) ICP-MS after 24 hr incubation with either RGDfK modified or untargeted GNRs (10 μg/ml). RGDfK-GNRs show increased binding and uptake relative to untargeted GNRs in both cell lines, however this difference was most significant (roughly 20-fold) with HUVECs.

FIG. 4 shows representative TEM images of RGDfK (A-C) and untargeted (D) GNRs in HUVECs after 24 hr incubation. Arrows point to location of GNRs within the cell. Some GNRs were found within multiple membranes (panel B) near the nucleus.

FIG. 5 shows RGDfK-GNR binding to HUVECs in: (A) absence and (B) presence of the αᵥβ₃ inhibitor echistatin (50 nM) at 4° C. for 2 hrs in binding buffer. Small green-yellow dots indicate presence of GNRs on the cell surface.

FIG. 6 shows (A) transmission electron micrograph of GNRs, and (B) light absorption profile of GNRs with SPR peak at 800 nm.

FIG. 7 shows intratumoral temperatures during PPTT or laser alone. Laser power=1.6 W/cm2 (A) and 1.2 W/cm² (B). Error bars represented as ±standard deviation.

FIG. 8 shows Evans blue dye (EBD) delivery thermal enhancement ratio (TER). **Indicates a statistically significant difference (p<0.01) by one-way analysis of variance (ANOVA). Error bars represented as ±standard deviation.

FIG. 9 shows the biodistribution of radiolabeled (¹²⁵I) HPMA copolymers in several organs.

FIG. 10 shows tumor accumulation of the untargeted and heat shock targeted HPMA copolymers after either treatment with hyperthermia (PPTT) or with no treatment (Control).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a cell cycle specific compound” includes mixtures of two or more such compounds, and the like.

“Optional” or “optionally” means that the subsequently described event or circumstance can or cannot occur, and that the description includes instances where the event or circumstance occurs and instances where it does not. For example, the phrase “optionally a cell cycle specific compound” means that the compound can or can not be included.

The term “bonded” refers to either chemical bonding (e.g., covalent or non-covalent bonding such as hydrogen bonding, dipole-dipole interactions, electrostatic, etc.) or the process of encapsulation or entrapment.

The term “polyalkylene group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide, and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyimino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, an oleate group or a palmitate group. A “lower alkyl” group is an alkyl group containing from one to six carbon atoms.

The term “alkyl group” also includes cycloalkyl groups. The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “amine group” as used herein represented by the formula —NRR′, where R and R′ are independently hydrogen or an alkyl or aryl group defined above.

The term “thioalkyl group” as used herein represented by the formula —SR, where R is an alkyl or aryl group defined above.

The term “alkoxy group” as used herein is represented by the formula —OR, where R is an alkyl or aryl group defined above. Examples of alkoxy groups include, but are not limited to, methoxy, ethoxy, and the like.

The term “residue” as used herein is a portion of a molecule or compound. For example, the residue having the formula Au—S-L-X means that at least one S-L-X group is bonded to the gold particle (Au). It is contemplated that multiple S-L-X groups can be bonded to the gold particle depending upon reaction conditions.

### I. Gold Particles

Described herein are gold particles that can be used to reduce tumor proliferation and treat cancer. In certain aspects, the gold particles can be modified in order to enhance selectivity and uptake of the particles by cancer cells. Each component used to make the gold particles and methods for making the gold particles is described below.

a. Gold Particle Precursors

The gold particles useful herein can be synthesized with very precise sizes and shapes. These constructs can take the form of spherical particles, rods (Giri S, Trewyn B G, Stellmaker M P, Lin V S Y. 2005. Stimuli-responsive controlled-release delivery system based on mesoporous silica nanorods capped with magnetic nanoparticles. Angew. Chem. Int. Edit. 44: 5038-5044); cages (Chen J, Wiley B, Li Z Y, Campbell D, Saeki F, Cang H, Au L, Lee J, Li X, Xia Y. 2005. Gold nanocages; Engineering their structure for biomedical applications. Adv. Mater. 17: 2255-2261; and discs (Ryan R O. 2008. Nanodisk: hydrophobic drug delivery vehicles. Expert Opin. Drug Del. 5: 343-351).

In one aspect, when the gold particle is a rod, the rod has a diameter from 5 nm to 500 nm. In other aspects, the rod has a diameter from 5 nm to 500 nm, 5 nm to 250 nm, 5 nm to 100 nm, 5 nm to 90 nm, 5 nm to 80 nm, 5 nm to 70 nm, 5 nm to 60 nm, 5 nm to 50 nm, 5 nm to 40 nm, 5 nm to 30 nm, 5 nm to 20 nm, or 8 nm to 18 nm. In one aspect, the rod has a length from 10 nm to 800 nm, 10 nm to 600 nm, 10 nm to 400 nm, 10 nm to 200 nm, 20 nm to 100 nm, or 25 nm to 80 nm. In a further aspect, the rod has a diameter of about 25 nm±5 nm, 30 nm±5 nm, 35 nm, 40 nm±5 nm, 45 nm±5 nm, 50 nm±5 nm, 55 nm±5 nm, 60 nm±5 nm, 65 nm±5 nm, 70 nm±5 nm, 75 nm±5 nm, or 80 nm±5 nm.

Not wishing to be bound by theory, if gold particles are exposed to wavelengths dictated by the particle's aspect ratio, then surface plasmon resonance may occur and the light energy is transformed into heat. This feature of the gold particles with respect to treating cancer will be described in detail below. In one aspect, when the gold particle is a rod, the rods have a higher intensity of plasmon resonance with narrower band-width. This feature is attractive in cancer treatment with respect to targeted tumor ablation. In one aspect, the gold particle has an aspect ratio of 1 to 50.

b. Linkers

In certain aspects, when the gold particle has a targeting group attached to it (referred to herein as a “modified gold particle”), the targeting group is attached to the surface of the gold particle via a linker. In general, it is desirable that the linker be biocompatible and non-toxic. The selection of the linker can be determined based on the desired properties of the linker and the end-use of the modified gold particles. For example, the linker can possess hydrophilic or hydrophobic properties. In one aspect, the linker can be a polymer such as a homopolymer, a copolymer, or a block copolymer. In another aspect, the linker can be a polyether group, polythioether group, polyimino group, polyester group, polyamide group, or a polyacrylate group.

In one aspect, the linker is a hydrophilic polymer. In this aspect, the hydrophilic polymer can be any water-soluble polymer useful in drug delivery. Examples of such polymers include polycaprolactone, polylactic acid, poly(lactide-co-glycolide), poly(hydroxybutyrate), poly(hydroxybutyrate-covalerate), polydioxanone, polyorthoester, polyanhydride, poly(glycolic acid), poly(glycolic acid-cotrimethylene carbonate), polyphosphoester, polyphosphoester urethane, poly(trimethylene carbonate), poly(iminocarbonate), cyanoacrylates, polyalkylene oxalates, polyphosphazenes, aliphatic polycarbonates, poly(amino acid)s (e.g., containing cysteine), cellulose, starch, dextran, hyaluronic acid, and collagen.

In one aspect, the hydrophilic polymer includes the polymerization product of N-(2-hydroxypropyl)methacrylamide (HPMA), hydroxyalkyl methacrylate (HEMA), hydroxyalkyl acrylate, N-vinyl pyrrolidone, N-methyl-3-methylidene-pyrrolidone, allyl alcohol, N-vinyl alkylamide, N-vinyl-N-alkylamide, acrylamides, methacrylamide, (lower alkyl)acrylamides and methacrylamides, hydroxyl-substituted (lower alkyl)acrylamides, methacrylamides, and any combination thereof.

In another aspect, the hydrophilic linker comprises a polymer of ethylene glycol, propylene glycol, or block co-polymers thereof. In one aspect, the linker is a poloxamer. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). Poloxamers useful herein are sold under the tradename Pluronic® manufactured by BASF. In another aspect, the hydrophilic linker is polyethylene glycol having a molecular weight from 100 to 30,000; 1,000 to 20,000; 2,000 to 10,000; 4,000 to 6,000, or about 5,000.

The linkers can be selected such that they possess functional groups that render the linker either degradable (e.g., biodegradable) or non-degradable. In one aspect, the linker can include a group that is pH sensitive and can be readily cleaved. An example of such a group includes, but is not limited to, a hydrazone (Etrych et al., J. Contr. Rel., 73, 2001, 89-102). In other aspects, the functional group can be an oligopeptide that is susceptible to enzymatic cleavage. For example, the oligopeptide can be GFLG, which is a lysosomally degradable bond (Etrych et al.). In other aspects, the linker can be sensitive to externally controlled stimuli. The stimuli can include, but are not limited to, the application or injection of enzymes, IR laser, UV or visible light, ultrasound, microwave, x-ray, temperature, and mechanical force. In one aspect, the linker can be polyesteramide copolymer based on ε-caprolactone 11-aminoundecanoic acid. In this aspect, the copolymer thermally degrades upon exposure to heat (Qian et al., Polymer Degradation and Stability, 81, 2003, 279-286). In another aspect, the linker can be a photodegradable polymer. For example, the polymer can be a poly(ether-ester) macromer. In one aspect, the poly(ether-ester) macromer is a polyethylene glycol capped with acrylate or methacrylate groups (see e.g., Nakayama et al., Acta Biomaterialia 7, 2011, 1496-1503; Kloxin et al., Science, 324, 2009, 59-63).

c. Targeting Groups

In certain aspects, a targeting group is attached to the gold particle via a linker. The targeting moiety can actively target either the tumor or the angiogenic blood vessel. Such targeting can be specific to antigens, growth factors, tumor promoters, essential hormones, enzymes or nutrients. The selection of the targeting group can vary depending upon the mechanism of localization into the tumor cells. For example, “active” mechanisms may encompass receptor mediated targeting of the modified gold particles described herein to a tumor cell. In the case of “passive” targeting, the targeting group can facilitate tumor localization by the EPR effect. Examples of targeting groups useful herein include, but are not limited to, monoclonal antibodies, peptides, somatostatin analogs, folic acid derivatives, lectins, polyanionic polysaccharides, or any combination thereof. In another aspect, the targeting group is a peptide having the sequence RGD or WIFPWIQL.

d. Preparation of Gold Particles

The gold particles described herein can be surface modified by a variety of techniques and sequences. In one aspect, the linker (L) can be mixed with the gold particles such that the linker forms a covalent bond with the gold surface. In this aspect, the linker possesses a group that can react with gold. For example, the linker can possess one or more thiol groups.

In one aspect, the gold particles include a residue having the formula I

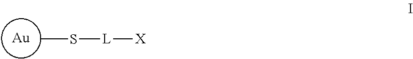

wherein
Au comprises a gold particle;
L comprises a linker; and
X comprises a functional group or a targeting group.

When the gold particles have a functional group at X, these are referred to herein as “unmodified gold particles.” In one aspect, the functional group X is any group capable of forming a covalent bond with a group present on a targeting group. In other aspects, X can be a group that can be further derivatized as desired. In one aspect, X is a hydroxyl group, an alkoxy group, a carboxy group, a carbonyl group, an amine group, or an amide group, an azide group, an imine group, a thiol group, a sulfonyl group, a thionyl group, a sulfonamide group, an isocyanate group, thiocyanate group, an epoxy group, a phosphate group, a silicate, a borate group. Conversely, when X is a targeting group, these particles are referred to herein as “modified gold particles.”

In one aspect, the gold particles are reacted with HS-PEG-Z to produce a residue having the formula IV

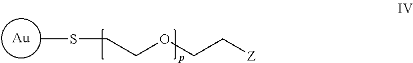

wherein
p is from 1 to 200,000; and
Z is a functional group.

In this aspect, the linker is poly(ethylene glycol) (PEG). In another aspect, Z is an alkoxy group such as methoxy, and p is from 20 to 2,000. Exemplary methods for preparing gold particles having the residue of formula IV are provided in the Examples.

In other aspects, when a targeting group is used, the targeting group can be attached to the linker first, and the linker-targeting group is subsequently attached to the gold particle. In one aspect, using this approach, the modified gold particle comprises a residue having the formula I

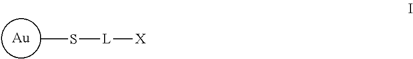

wherein
Au comprises a gold particle;
L comprises the linker as described herein; and
X comprises the targeting group as described herein,

In another aspect, the residue comprises the structure II

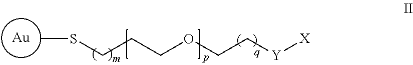

wherein
m is 1 to 100, 1 to 50, 1 to 25, 1 to 10, or 1 to 5;
p is from 1 to 200,000; 1 to 100,000; 1 to 50,000, 5 to 25,000, 10 to 10,000; 15 to 5,000; or 20 to 2,000;
q is from 0 to 100; 1 to 50, 1 to 25, 1 to 10, or 1 to 5;
Y is oxygen, sulfur, a substituted or unsubstituted amino group, a carbonyl group, an ester group, or an amide group; and
X is a targeting group.

In one aspect, the modified gold particle has a residue having formula II, wherein m is 2 and q is 1. In a further aspect, the modified gold particle has a residue having formula III

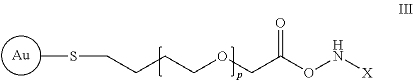

wherein
p is from 1 to 200,000; and
X is a targeting group.

In this aspect, a compound having the formula V is reacted with gold particles to produce formula III.

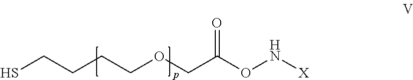

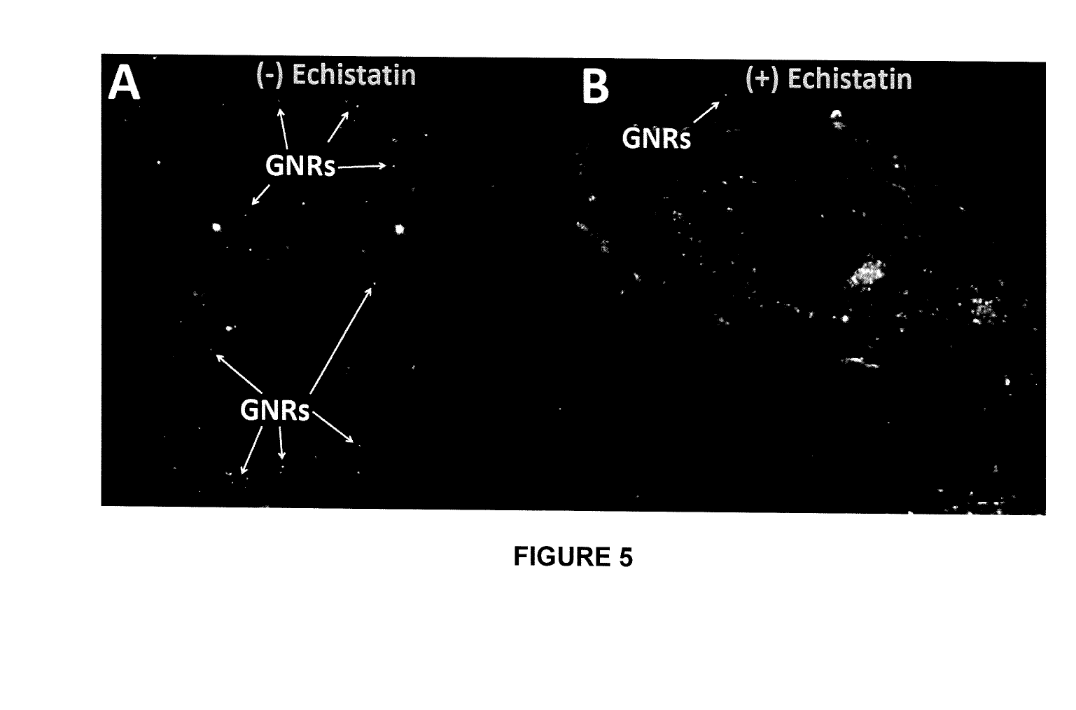

An exemplary procedure for making modified gold particles having the residue I-III can be found in FIG. 5 and the Examples.

### II. Pharmaceutical Compositions

The gold particles described herein can be formulated into a variety of pharmaceutical compositions depending upon the mode of administration. Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, poly(ethylene glycol), and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

One advantage of the gold particles described herein is that they are stable in aqueous solution. In other words, the gold particles do not agglomerate and, thus, precipitate out of solution. In certain aspects, the gold particles form colloidal suspensions in aqueous medium. This is a very important feature with respect to the administration of the particles in aqueous medium such as, for example, intravenous injection. Experimental details regarding the stability of the particles are provided in the Examples.

The pharmaceutical composition can be administered in a number of ways depending on whether local or systemic treatment is desired, and on the area to be treated. Administration can be topically, including ophthalmically, vaginally, rectally, intranasally. Administration can also be intravenously or intraperitoneally. In the case of contacting cancer cells with the compounds described herein, it is possible to contact the cells in vivo or ex vivo.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined Particularly preferred compositions and methods are disclosed in the Examples herein, and it is understood that these compositions and methods, while not necessarily limiting, can be performed with any of the compositions and methods disclosed herein.

### III. Methods of Use

The gold particles described herein (modified or unmodified) can reduce or prevent tumor cell proliferation and, thus, be useful in treating cancer. The gold particles can be used alone or in combination with other anti-cancer agents. As will be discussed in detail below, the gold particles can enhance the ability of anti-cancer agents to penetrate cancer cells. Thus, the gold particles behave synergistically with other cancer treatments.

In one aspect, a method for treating cancer in a subject comprises:
(1) administering to a subject having a tumor (a) any of the gold particles described herein and (b) at least one-anti-cancer agent directly or indirectly bonded to the macromolecule and/or unbound to the macromolecule;
(2) exposing the tumor to light for a sufficient time and wavelength in order for the gold particles to achieve surface plasmon resonance.

In another aspect, a method of reducing or preventing tumor cell proliferation comprises
(1) contacting the tumor cells with an effective amount of (a) any of the gold particles described herein and (b) at least one-anti-cancer agent directly or indirectly bonded to the macromolecule and/or unbound to the macromolecule; and
(2) exposing the cells to light for a sufficient time and wavelength in order for the gold particles to achieve surface plasmon resonance.

The selection of the macromolecule can vary depending upon, among other things, the anti-cancer agent selected and the type of cancer to be treated. In one aspect, the macromolecule is capable of passively targeting tumor cells and tissues to reduce or prevent tumor cell proliferation. For example, the macromolecules can accumulate inside a tumor via the enhanced permeability and retention (EPR) effect. EPR is the passive accumulation of substances such as macromolecular conjugates inside a tumor. This property is associated with a compound's affinity for accumulating in tumor tissue much more rapidly than in normal tissues. For tumor cells to grow quickly, blood vessel production must be stimulated. Newly formed tumor blood vessels are usually abnormal in form and architecture. For example, tumor blood vessels display poorly-aligned endothelial cells with wide fenestrations, and tumor cells and tumor tissues generally lack effective drainage. Due to these defects and the presence of tumor vascular permeability factor, bradykinin, and tumor necrosis factor, tumor vasculature permits large macromolecules to enter tumor tissue more quickly than into normal tissues. In addition, poor lymphatic drainage and high hydrostatic pressure results in delayed clearance and longer retention of macromolecules within tumors.

A variety of macromolecules are suitable for use herein and generally include any macromolecule that is biocompatible, e.g., non-toxic and non-immunogenic. In certain aspects, the macromolecule is synthetic to enable the molecular weight range to achieve a size appropriate for enhanced trans-endothelial permeation and retention at a tumor site and for renal filtration.

The molecular weight of the macromolecule can vary. By varying the molecular weight of the macromolecule, it is possible to modify the blood circulation lifetime and body distribution of the compound, in particular its enhanced endothelial extravasation and retention at the tumor. The polydispersity of the macromolecule is also a factor in circulation lifetime and distribution. In one aspect, the macromolecule has a molecular weight of between about 1 kD to 5,000 kD, 5 kD to 500 kD, or 10 kD to 200 kD.

The size (hydrodynamic volume) of the macromolecule can vary. By varying the size (hydrodynamic volume) of the macromolecule, it is possible to modify the blood circulation time and body distribution of the compound, in particular its enhanced endothelial extravasation and retention at the tumor. The polydispersity of the macromolecule is also a factor in circulation time and distribution. In one aspect, the macromolecule has a hydrodynamic volume of between about 0.1 nm (nanometer) to 5,000 nm, 1 nm to 1000 nm, or 5 nm to 500 nm.

Macromolecules suitable for in vivo administration include, but are not limited to, dextran, dextrin, hyaluronic acid, chitosan, polylactic/glycolic acid (PLGA), poly lactic acid (PLA), polyglutamic acid (PGA), polymalic acid, polyaspertamides, poly(ethylene glycol) (PEG), poly-N-(2-hydroxypropyl)methacrylamide (HPMA), poly(vinylpyrrolidone), poly(ethyleneimine), poly(amido amine) (linear), and dendrimers comprising poly(amido amine), poly(propyleneimine), polyether, polylysine, or any combination thereof. In another aspect, the macromolecule includes N-alkyl acrylamide macromolecules such as homopolymers and copolymers prepared from monomers of the acrylamide family including acrylamide, methacrylamide and hydroxypropylacrylamide.

In one aspect, the macromolecule can be a dendrimer. Dendrimers are multi-functional, symmetric, nano-sized macromolecules useful as delivery devices. They are characterized by a unique tree-like branching architecture and a compact spherical shape in solution. Their potential as drug carriers arises from the large number of arms and surface groups that can be functionalized to immobilize drugs, enzymes, targeting moieties, or other bioactive agents. The molecular weight of the dendrimer can be adjusted with appropriate linkers and drugs. The use of dendrimers herein can provide several unique features with respect to the delivery of drugs, including (ii) a dendrimer's architecture can dramatically influence pharmacokinetics; (iii) the addition of certain groups such a, for example, PEGylation, increases water solubility and dendrimer size, and can lead to improved retention and biodistribution characteristics; (iv) therapeutic agents can be internalized in the void space between the periphery and core, or covalently attached to functionalized surface groups; and (v) targeting moieties can be bound to the dendrimer's surface (discussed below). In one aspect, the dendrimer includes poly generation 1, 1.5, 2, 2.5. 3, 3.5, 4, 4.5, 5, 5.5, 6, 6.5, 7, or 7.5, 8, 8.5, 9, 9.5, or 10. The dendrimer can be produced from a variety of different building blocks. In one aspect, the macromolecule is poly(amido amine) (PAMAM), diaminobutane (DAB), diaminoethane (DAE), melamine based or poly (ethylene glycol) derived.

In another aspect, the macromolecule can be a water soluble drug delivery system including an inert synthetic polymeric carrier. In this aspect, the macromolecule is 5.0 to 99.5 mol % monomeric units including, but not limited to, N-(2-methylpropyl)methacrylamide, N-(2-methylethyl)methacrylamide, N-isopropyl methacrylamide, N,N-dimethacrylamide, N-vinylpyrrolidone, vinyl acetate, 2-methacryloxyethyl glycoside, acrylic acid, methacrylic acid, vinylphosphonic acid, styrenesulfonic acid, malic acid, 2-methacryloxyethyltrimethylammonium chloride, 2-methacrylamidopropyltrimethylammonium chloride, methacryloylcholine methyl sulfate, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimines, allylamine, or any combination thereof. Thus, the macromolecule can be a homopolymer or copolymer.

The anti-cancer agent can be directly or indirectly bonded to the macromolecule. The term “indirectly bonded” as used herein is defined when the anti-cancer agent is attached to the macromolecule via a linker. Any of the linkers described above can be used in these aspects. Conversely, the term “directly bonded” as used herein is when the anti-cancer agent is attached to the macromolecule without a linker. In the case of the anti-cancer agent, the agent is generally covalently attached to the linker (i.e., indirect bonding) or macromolecule (i.e., direct bonding). In general, the macromolecule has one or more functional groups that can form a covalent bond with the linker. The linker used in these aspects can be the same or different linker used in the preparation of the gold particles described above.

The nature and selection of the linker can vary. As discussed above, the linker can include one or more functional groups that are capable of forming covalent bonds with the macromolecule and anti-cancer agent. The functional groups generally contain heteroatoms such as oxygen, nitrogen, sulfur, or phosphorous. Examples of functional groups present on the linker include, but are not limited to, hydroxyl, carboxyl (acids, esters, salts, etc.), amide, amino (substituted and unsubstituted), thiol, acyl hydrazones and the like.

The selection of the linker can also vary one or more properties of the compound. For example, the linker can be a group that modifies the hydrophobic or hydrophilic properties of the compound. An example of this is poly(ethylene glycol) (PEG). PEG is generally a hydrophilic material, and by varying the molecular weight of PEG, the hydrophilic properties of the compound can be modified. In one aspect, PEG has a molecular weight from 50 D to 200 kD, 50 D to 100 kD, 50 D to 50 kD, or 50 D to 20 kD. PEG can also be used to produce biocompatible copolymers such as, for example, (PEG-diacrylate (PEGDA), PEG-dimethacrylate (PEGDM), PEG-diacrylamide (PEGDAA), or PEG-dimethacrylamide (PEGDMA). Although PEG and related compounds are suitable as a linker herein, the linker can be other groups such as, for example, short chain (e.g., C₁-C₆) ethers, esters, amines, amides, and the like.

In other aspects, the linker can be an oligopeptide sequence, an amino acid, or amino acid sequence. For example, amino acids can contain amino, thiol, and carboxyl groups that can form non-covalent bonds with anti-cancer agents such as Z elements, which are discussed in detail below. In this aspect, the high Z element is non-covalently bonded to the linker via coordinate covalent bonding. The functional groups present on the amino acid or oligopeptide also permit attachment of the linker to the macromolecule. In one aspect, the amino acid or oligopeptide linkers are 1 to 6 amino acids in length. In this aspect, the amino acid or oligopeptide linkers include, but are not limited, to the following sequences: Gly-Ileu-Phe, Gly-Val-Phe, Gly-Gly-Phe, Gly-Gly-Phe-Phe, Gly-Ileu-Tyr, Phe, Gly, Gly-Gly, Ala, Ser, Gly-Phe, Gly-Leu-Phe, Gly-Phe-Phe, Gly-D-Phe-Phe, Ala-Gly-Val-Phe, Gly-Gly-Val-Phe, Gly-Phe-Tyr, Gly-□-Ala-Tyr, Gly-Leu, Gly-Phe-Leu-Gly, Gly-Phe-Gly, Gly-Gly, or any combination thereof. The oligopeptide can be linked by an amine, amide, ester, ether, thioether, acyl hydrazones, carbonate, carbamate, disulfide linkage and alike. In other aspects, the macromolecule can be an amphiphile. Amphiphiles useful herein are compounds possessing hydrophilic and lipophilic groups capable of forming micelles or liposomes. The amphiphiles should be biocompatible such that they possess minimal toxicity. Amphiphiles useful herein for preparing liposomes and micelles include homopolymers, copolymers, block-copolymers produced from biocompatible and biodegradable materials. Examples of such macromolecules include, but are not limited to, poly(amino acid)s; polylactides; poly(ethyleneimine)s; poly(dimethylaminoethylmethacrylate)s, copolymers of polyethyelene glycol and hydroxyalkyl acrylates and acrylamides (e.g., N-(2-hydroxypropyl)methacrylamide), PEG-α-poly(α-amino acids), poly(L-lactic acid)-poly(ethylene glycol) block copolymers, or poly(L-histidine)-poly(ethylene glycol) block copolymers. Thus, in this aspect, the macromolecule can entrap anti-cancer agents without any bonding between the macromolecule and the anti-cancer agent.

In one aspect, the amphiphile is a poloxamer. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF.

In other aspects, the amphiphile can be a lipid such as phospholipids, which are useful in preparing liposomes. Examples include phosphatidylethanolamine and phosphatidylcholine. In other aspects, the amphiphile includes cholesterol, a glycolipid, a fatty acid, bile acid, or a saponin.

The selection of the anti-cancer agent can vary as needed. The anti-cancer agent can be cell cycle specific compounds or non-cell cycle specific compounds. Although not always the case, the anti-cancer agent kills cells via a different mechanism than the high Z elements group (i.e., generation of Auger electrons). Examples of anti-cancer agents useful herein include, but are not limited to, abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gem tuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, or an analog thereof. Analogs of any of the anti-cancer agents are also contemplated herein. For example, different derivatives of the agent can be used.

In other aspects, the anti-cancer agent can be a variety of different high Z elements that produce Auger electrons and can be used herein. In one aspect, the high Z elements group includes iodine, lutenium, hafnium, tantalum, tungsten, rhenium, osmium, iridium, platinum, gold, thallium, lead, bismuth, radon, franceium, or any combination thereof. In another aspect, the high Z element group is a platinum containing chemotherapeutic agent such as, for example, cisplatin, carboplatin, oxiplatin, nedaplatin, lipoplatin, satraplatin, ZD0473, BBR3464, SPI-77, or any combination thereof. In certain aspects, the macromolecule can have two or more anti-cancer agents bonded to it (e.g., a Z element and a pharmaceutical such as geldamycin).

In certain aspects, the compounds described can have one or more targeting groups directly or indirectly bonded to the macromolecule. In the case when the targeting group is bonded to the macromolecule, any of the linkers described herein can be used. The selection of the targeting group can vary depending upon the mechanism of localization into the tumor cells. For example, “active” mechanisms may encompass receptor mediated targeting of the compounds described herein to a tumor cell. In the case of “passive” targeting, the targeting group can facilitate tumor localization by the EPR effect. Examples of targeting groups useful herein include, but are not limited to, monoclonal antibodies, peptides, somatostatin analogs, folic acid derivatives, lectins, polyanionic polysaccharides, or any combination thereof. In one aspect, the targeting group is a cyclic RGD peptide such as, for example, (1) RGD4C (Ala-Cys-Asp-Cys-Arg-Gly-Asp-Cys-Phe-Cys-Gly), (2) RGE4C (Ala-Cys-Asp-Cys-Arg-Gly-Glu-Cys-Phe-Cys-Gly), or (3) RGDfK (Arg-Gly-Asp-D-Phe-Lys). In another aspect, the targeting group is a peptide having the sequence RGD or WIFPWIQL.

In other aspects, the macromolecule can have one or more polydentate ligands. A “polydentate ligand” is a ligand that can bind itself through two or more points of attachment to a metal ion through, for example, coordinate covalent bonds. In one aspect, the polydentate ligand can chelate with metal ions such as gadmium, which can be used as a contrast agent. Examples of polydentate ligands include, but are not limited to, diethylenetriaminepentaacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

In another aspect, the macromolecule is a copolymer prepared from N-(2-hydroxypropyl)methacrylamide, where geldanamycin is indirectly bonded to the macromolecule by an oligopeptide, and a targeting group having the sequence WIFPWIQL is bonded to the macromolecule.

In one aspect, the gold particles described herein can reduce or prevent tumor cell proliferation alone or in combination with other anti-cancer agents. The tumor or cancer cells can be contacted with the particles described herein in vitro, in vivo, or ex vivo. In one aspect, when the application is in vivo, the compound can be administered to a subject by techniques known in the art. For example, the compound can be administered intraveneously to the subject. Alternatively, the compound can be injected directly into the tumor. The number of times the compound is administered to the subject and the intervals of administration can vary depending upon the subject and the dosage of compound.

In one aspect, the gold particles are administered first followed by the administration of the macromolecule. In another aspect, the macromolecule is administered first followed by the administration of the gold particles. In other aspects, the gold particles and the macromolecule are administered simultaneously. In these aspects, the gold particles and the macromolecule can be administered intraveneously. In one aspect, a kit comprising the gold particles and the macromolecule is contemplated. In another aspect, the gold particles and macromolecule can be formulated into one composition.

After contacting the cancer cells as described above, the tumor or cancer cells are exposed to light for a sufficient time and wavelength in order for the gold particles to achieve surface plasmon resonance. Not wishing to be bound by theory, plasmonic gold particles with a large light extinction profile can be used as nano antennas for photothermal ablative therapy. Able to generate intratumoral heat, a minimally invasive laser light source whose wavelength overlaps with the localized surface plasmon resonance (SPR) peak can cause hyperthermia and at higher temperatures extensive vascular damage. In one aspect, plasmonic photothermal therapy (PPTT) can induce tumor hyperthermia, increase tumor penetration of macromolecular therapeutics at controlled temperatures, and also act as an effective antivascular therapy. In this aspect, macromolecules possessing anti-cancer agents can weaken the tumor leaving the malignancy more susceptible to photothermal damage. When used synergistically, these two approaches may dramatically reduce the amount of laser energy required to kill the tumor, maximize tumor kill and minimize toxicity. In one aspect, the tumor is exposed to light produced from a laser diode light source comprising a dose from 0.25 to 4 W/cm² for a duration of 1 to 60 minutes.

In one aspect, the tumor or tumor cells are exposed to light for a sufficient time and wavelength in order to elevate the temperature inside the tumor or tumor cells from 40° C. to 50° C., 42° C. to 48° C., or 43° C. to 47° C. In another aspect, the tumor or tumor cells are exposed to light for a sufficient time and wavelength in order to elevate the temperature inside the tumor or tumor cells from 42° C. to 43° C. Hyperthermia enabled drug delivery has several limitations. There exists a very narrow window where increased blood perfusion and permeability is observed without severe vascular damage. Therefore, using standard techniques of inducing hyperthermia in the clinic while maintaining a tumor temperature within this therapeutic window is difficult. Also, non-specific heating of surrounding healthy tissue may increase the probability of drug delivery within those regions where undesired toxicity is likely to occur. PPTT has the potential to partially address these issues. Control of laser beam power and alignment may enable clinicians to precisely control thermal dose in a directed way. Additionally, PPTT represents a targeted approach to hyperthermia.

The methods described herein can be used to treat a variety of different tumors and cancers including, but not limited to, a breast tumor, a testicular tumor, an ovarian tumor, a lymphoma, leukemia, a solid tissue carcinoma, a squamous cell carcinoma, an adenocarcinoma, a sarcoma, a glioma, a blastoma, a neuroblastoma, a plasmacytoma, a histiocytoma, an adenoma, a hypoxic tumor, a myeloma, a metastatic cancer, bladder cancer, brain cancer, nervous system cancer, head and neck cancer, squamous cell carcinoma of head and neck, kidney cancer, lung cancers including small cell lung cancer and non-small cell lung cancer, neuroblastoma/glioblastoma, ovarian caner, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer, colorectal cancers, prostatic cancer, or pancreatic cancer.

### EXAMPLES

The following prophetic examples are put forth so as to provide those of ordinary skill how to make exemplary compounds described herein.

I. Synthesis and Characterization of Modified Gold Particles with Targeting Group

### Methods

### GNR Synthesis and Characterization

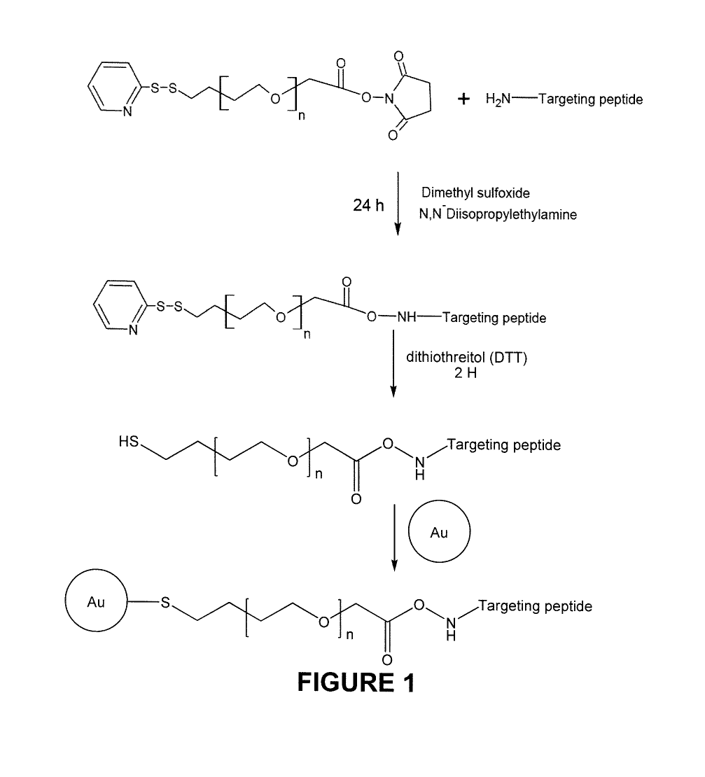

FIG. 1 provides a reaction scheme for producing GNRs with a targeting group. GNRs were synthesized using the seed-mediated growth method. Optimization of silver nitrate content and seed amount yielded GNRs with an aspect ratio such that the surface plasmon resonance (SPR) peak was between 800-810 nm. GNRs were then centrifuged (6,000 rcf, 30 minutes) and washed three times with deionized (DI) water to remove excess hexadecyltrimethylammonium bromide (CTAB). For the untargeted GNRs, poly(ethylene glycol) (PEG) (methoxy-PEG-thiol, 5 kD, Creative PEGWorks #PLS-604) was added to the GNR suspension (optical density (OD)=10) at a final PEG concentration of 100 μM and stirred for 1 hour. This was done to reduce the extent of protein adsorption and improve circulation time. The PEG-GNR suspension was then thoroughly dialyzed (3.5 K MWCO, Spectrum Labs #132594) and sterile filtered. Finally, the GNR suspension was centrifuged, washed three times with DI water to remove unreacted PEG and concentrated to a final concentration of 1.2 mg/ml (OD=120). Final product was stored at 4° C. for a maximum of 2 months due to polymer shedding over time before use.

Targeted GNRs were synthesized by first reacting ortho-pyridyl-disulfide-PEG-succinimidyl ester (OPSS-PEG-NHS, 5 kD, Creative PEGWorks #PHB-997, 50 mg) with RGDfK (New England Peptide, Inc., 6 mg) in anhydrous DMSO (5 ml) and three drops of diisopropylethylamine (DIPEA) while stirring for 24 hours at room temperature. Dithiothreitol (DTT, 7 mg) was then added to the reaction mixture and stirred for an additional 2 hours to reduce the disulfide bond and obtain a free thiol at the end of the PEG-RGDfK polymer. The mixture was then dialyzed (3.5 K MWCO, Spectrum Labs #132594) and lyophilized to obtain the final product. Finally, the thiol-PEG-RGDfK polymer was grafted to the gold surface in the same way as the untargeted GNR conjugate.

GNR size and shape were measured by transmission electron microscopy (TEM, FEI Tecnai T12) after drop-casting the GNR suspension onto a copper grid. The GNR light absorption profile was measured before and after PEGylation using a spectrophotometer (Jasco V-650) and the stability of these conjugates was measured the same way after 30 minutes in 3.5% NaCl. GNR concentration was determined by inductively coupled plasma mass spectrometry (ICP-MS, Agilent 7500ce) against a gold and internal (irradium) standard. The zeta potential of the conjugates was measured in DI water by measuring the particle's electrophoretic mobility using laser doppler velocimetry (Malvern Instruments Zetasizer Nano-ZS). Finally the RGDfK content on the gold was determined by amino acid analysis (University of Utah Core Research Facilities, Salt Lake City, Utah).

### Cell Culture

The binding and uptake was evaluated for targeted (RGDfK) and untargeted GNRs in two cell lines obtained from ATCC (Manassas, Va.); DU145 prostate cancer and human umbilical vein endothelial cells (HUVEC). DU145 cell lines were cultured in Eagle's Minimum Essential Medium with Earle's Balanced Salt Solution (ATCC) supplemented with 10% (v/v) fetal bovine serum (FBS) (Thermo Scientific HyClone, Logan, Utah). HUVEC cell lines were cultured in Clonetics Endothelial Cell Basal Medium-2 supplemented with 2% FBS, hydrocortisone, hFGF-B, VEGF, R3-IGF-1, ascorbic acid, hEGF, GA-1000 and heparin (Lonza EGM-2 BulletKit). Cell lines were cultured at 37° C. in 100% humidity with 5% CO₂. All cells were kept within logarithmic growth and while DU145 cells were kept under 20 passages, HUVEC cells were discarded after seven.

### Dark Field Microscopy

Cells were plated on sterile cover slips coated with fibronectin and allowed to grow until 50% confluent. The media was then replaced with either fresh media or media containing either the RGDfK or untargeted GNRs (10 μg/ml). Cells were allowed to incubate for 24 hours followed by aspiration of GNR containing media and three washing steps with phosphate buffered saline (PBS) followed by fixation for 10 minutes with 4% paraformaldehyde before mounting to a slide with mounting medium. To detect association (binding and uptake) of GNRs with the cells, slides were then imaged with an Olympus BX41 microscope coupled to the CytoViva 150 Ultra Resolution Imaging (URI) System (CytoViva Inc., Auburn, Ala.) using 100× oil objective. A DAGE XLM (DAGE-MTI, Michigan City, Ind.) digital camera and software was used to capture and store images.

### ICP-MS

To quantify binding and uptake, cells were plated in 24-well plates and allowed to grow to 70% confluency. After incubation with GNRs and washing with PBS as described above, cells were lysed with 100 mM sodium hydroxide for 20 minutes while shaking and the protein content for each well was determined using a bicinchoninic (BCA) protein assay (Micro BCA Protein Assay Kit, Thermo Scientific Inc., Rockford, Ill.). The lysate was then transferred to Teflon vials, digested and evaporated three times with fresh trace-metal grade aqua regia, then resuspended in 5% trace-metal grade nitric acid before being analyzed by ICP-MS for gold content quantification against a gold and internal standard. All groups were done in triplicates.

### TEM

For visualization of uptake by TEM, cells were grown to 50% confluency on fibronectin coated ACLAR® plastic films before 24 hr incubation with GNRs. Cells were then washed three times with PBS and fixed with 2.5% glutaraldehyde and 1% paraformaldehyde in 0.1M sodium cacodylate with sucrose and calcium chloride. Samples were then dehydrated with washes of increasing concentrations of ethanol and embedded in an epoxy resin before sectioning with an ultramicrotome. All samples were then imaged using a FEI Tecnai T12 microscope (University of Utah Core Research Facilities, Salt Lake City, Utah).

### Competitive Inhibition of Binding

Confirmation of RGDfK-GNR specificity to αᵥβ₃ integrins was performed by competitive inhibition of binding with echistatin. In brief, HUVEC cells were grown to 50% confluency on fibronectin coated cover slips. The media was then removed and replaced with cold binding buffer (20 mmol/L Tris, pH 7.4, 150 mmol/L NaCl, 2 mmol/L CaCl₂, 1 mmol/L MgCl₂, 1 mmol/L MnCl₂, 0.1% bovine serum albumin) containing RGDfK-GNRs (10 μg/ml) and HUVECs were co-incubated at 4° C. for 2 hours with or without 50 nM echistatin (Sigma-Aldrich). Cells were then washed three times with cold binding buffer, mounted to a slide and imaged by high-resolution dark field microscopy.

### Results

### GNR Synthesis and Characterization

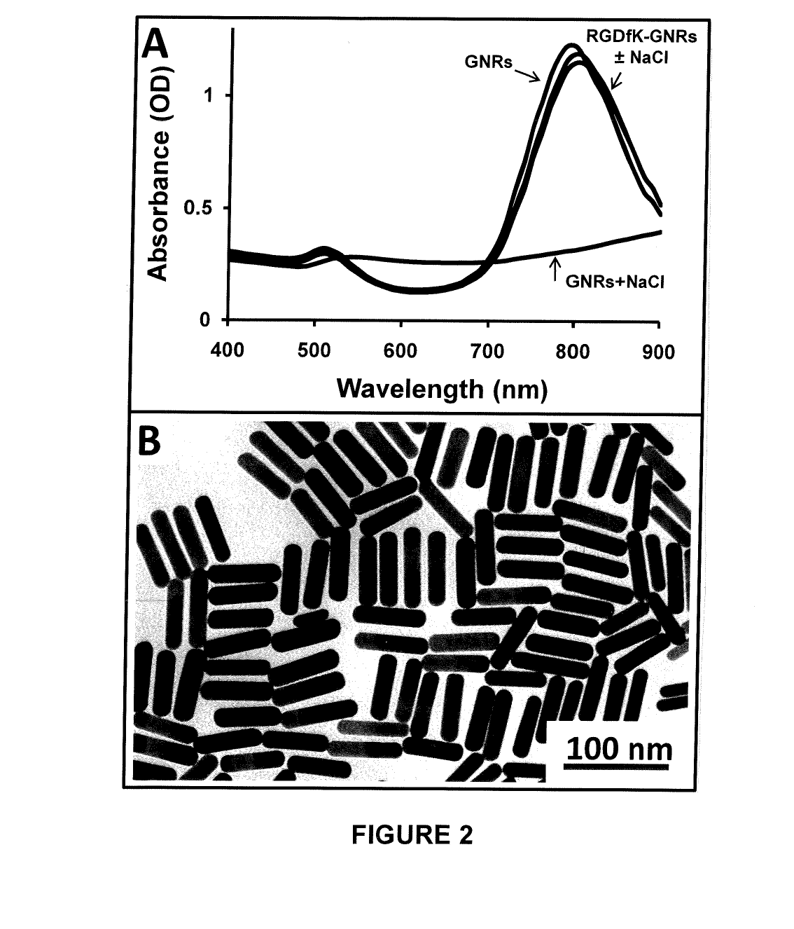

GNRs were synthesized with an SPR peak at 800 nm corresponding to a size of 60.5×15.0±6.4×2.0 nm with an aspect ratio equal to 4.0 (FIG. 2, Table 1). After PEGylation, with or without RGDfK, there was minimal change in absorption profile and the nanoparticles had strong stability in the presence of 3.5% NaCl. Zeta potential measures indicate that while the untargeted (methoxy terminated) GNRs had a slight negative charge (−10.0 mV), the RGDfK-GNRs had a strong negative charge (−44.1 mV). Amino acid analysis confirmed the presence of RGDfK on the targeted GNRs with a concentration equal to 5.6×10⁻¹¹ M_{RGDfK}/μg_{Au}.

TABLE 1
Physiochemical characteristics of GNRs

| Size (nm) | SPR | | Charge | Peptide Content |
|---|---|---|---|---|
| 60.5 × 15.0 ± | 800 nm | Untargeted | −10.0 mV | NA |
| 6.5 × 2.0 | | Targeted | −44.1 mV | 5.6 × 10⁻¹¹ |
| | | | | M_{RGDfk}/μg (Au) |

### Binding and Uptake by Dark Field Microscopy and ICP-MS

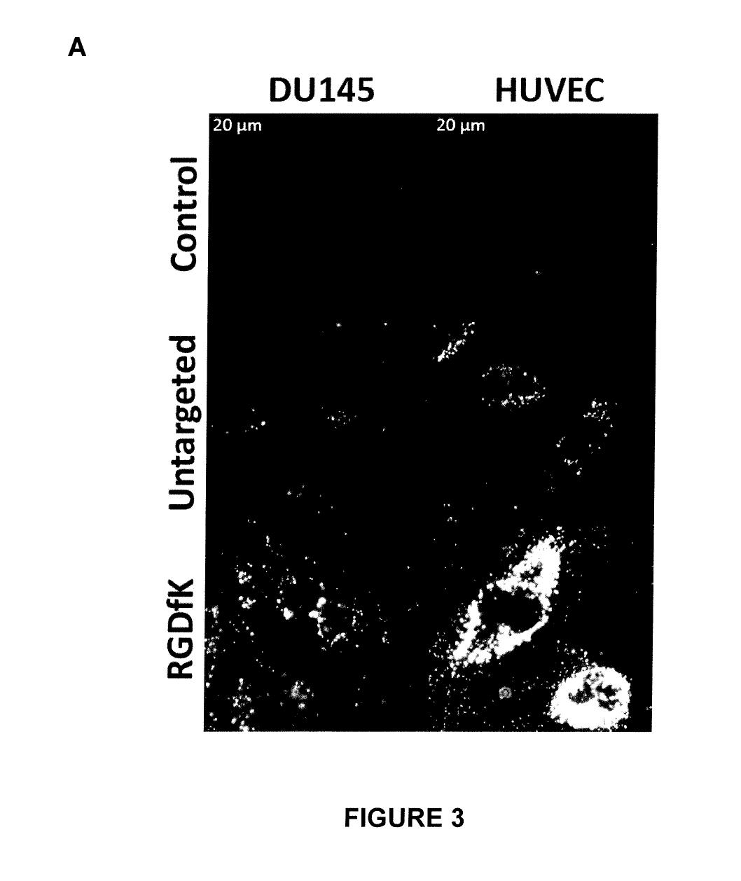

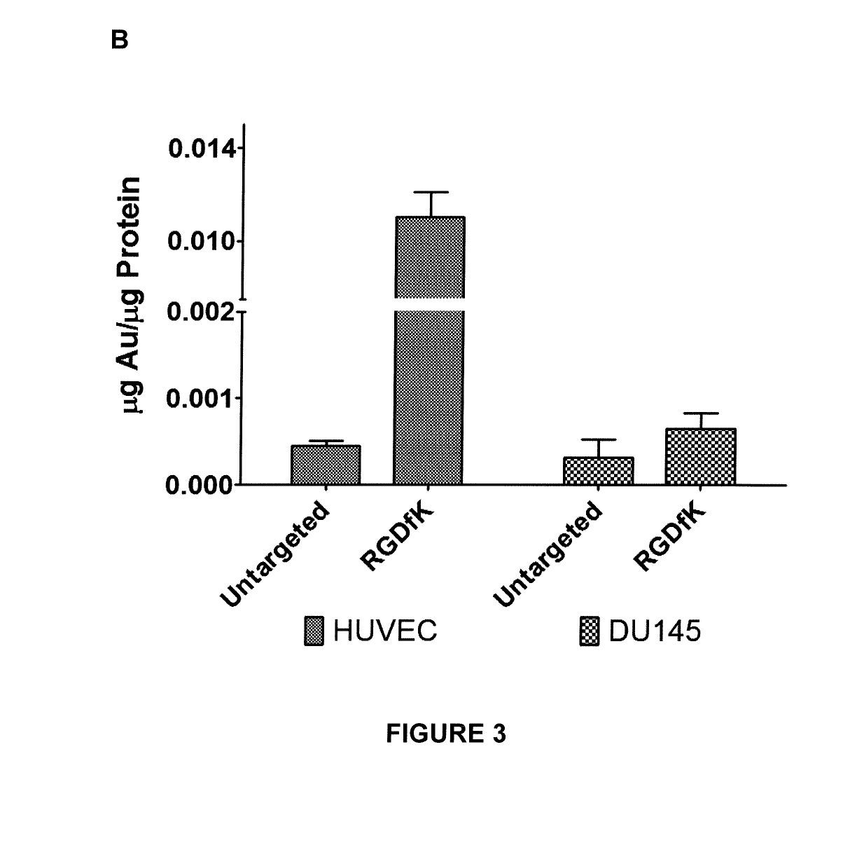

Because GNRs scatter light to a very high extent, the binding and uptake of both the untargeted and targeted (RGDfK) GNRs were visualized by high-resolution dark field microscopy (FIG. 3A). Captured images show that GNRs were associated with cultured cells to a different extent and do not affect overall cell morphology and the confluency of the culture. The untargeted GNRs showed some binding and uptake in both cell lines tested (DU145 and HUVEC). Internalized GNRs were primarily located in the perinuclear regions of the cells. Similarly, it appeared that the RGDfK-GNRs had slightly more uptake in DU145 cells than the untargeted GNRs, though this difference was not statistically significant after quantification by ICP-MS (FIG. 3B). After incubation of the targeted (RGDfK) GNRs with HUVECs however, significant binding and uptake was observed. ICP-MS analysis revealed that these binding and uptake events were roughly 20-fold higher for the targeted GNRs than the untargeted GNRs for HUVECs (FIG. 3B).

### Binding and Uptake by TEM

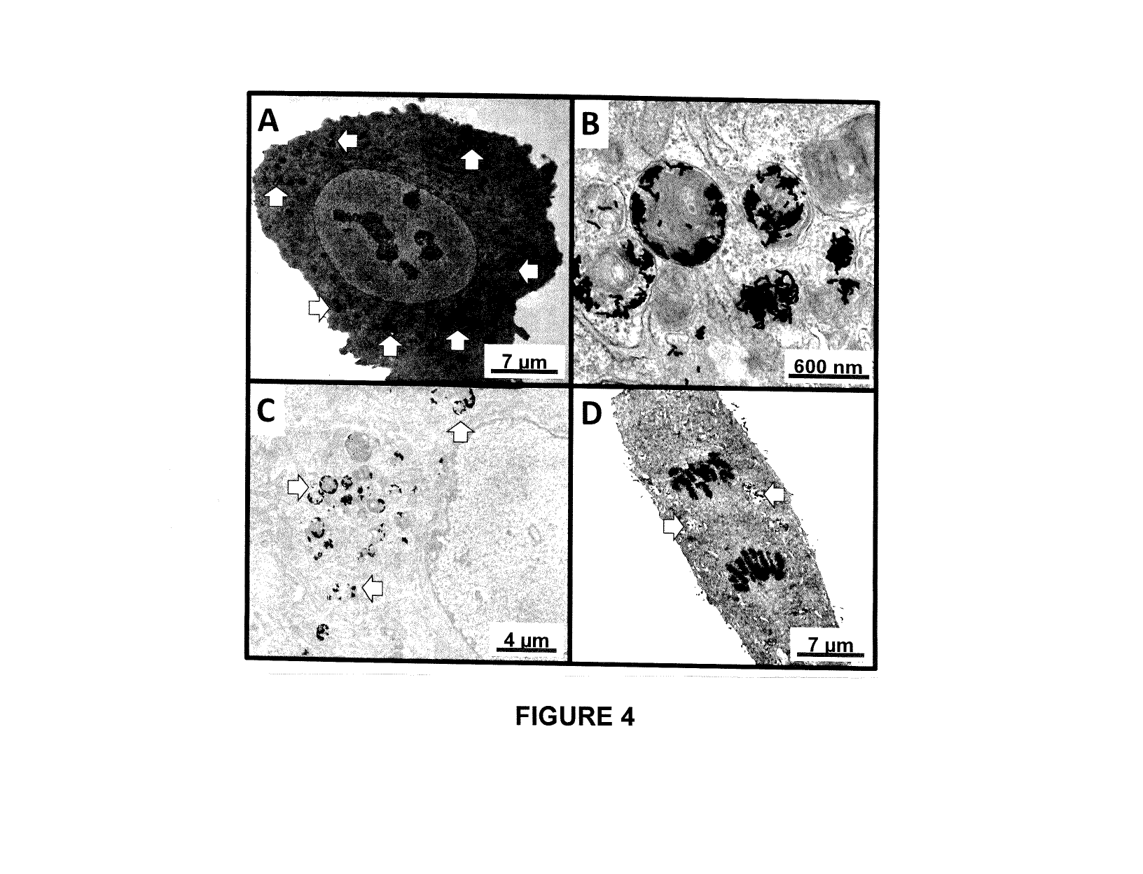

GNR uptake patterns by cells were typically as agglomerates and within membrane enclosed vacuoles (FIG. 4). In some cases, the agglomerates were found in vesicles with multiple membranes suggesting possible association within the endoplasmic reticulum (ER). Despite significant uptake and GNR loading within the cells no obvious evidence of intracellular structure and organelle damage was observed. These observations and the fact that there were no visible changes of cell culture confluence after incubation with GNRs, provide evidence related to the overall biocompatibility of the nanoparticles. Though in all cases uptake was observed by cells, the uptake of RGDfK-GNRs in HUVECs was significantly higher than that of any other cell line and particle combination.

### Competitive Inhibition of Binding

As echistatin is known to bind to αᵥβ₃ cell adhesion integrins with very high affinity, competitive binding inhibition of the RGDfK targeted receptors with this protein is possible. Incubation of HUVECs with RGDfK-GNRs at 4° C. for 2 hours in binding buffer alone resulted in some GNR binding along the cell's surface as visualized as small green-yellow dots observable by dark field microscopy (FIG. 5). To confirm the specificity of this binding, co-incubation with echistatin (50 nM) resulted in almost complete inhibition of GNR binding to the cell's surface. In only a few cases were GNRs found on the cell's surface which is in sharp contrast to those cells treated with RGDfK-GNRs alone where the nanoparticles were easily identifiable.

II. Evaluation of Modified Gold Particles in Combination with Macromolecules Having Anti-Cancer Agents

### Methods

GNRs were synthesized with an SPR peak between 800-810 nm by the seed-mediated growth method. A seed solution was first made by reduction of gold chloride (0.50 mM) in cetyltrimethylammonium bromide (CTAB) (0.20 M) with sodium borohydride (10 mM). A small amount of the seed solution was added to a growth solution containing gold chloride (1.0 mM), CTAB (0.20 M) and silver nitrate (4.0 mM) to form rods in the presence of ascorbic acid (78.8 mM). Resulting GNRs were sized by transmission electron microscopy (TEM) and the SPR peak was measured spectrophotometrically. After washing by centrifugation to remove excess CTAB, CH₃-PEG-SH (5 kD, 100 μM) was added and allowed to react with the gold surface for one hour followed by dialysis (3,500 Da cutoff). Resulting solution was washed and concentrated by centrifugation to remove unreacted PEG. Stability was assessed in 3.5% NaCl to confirm PEGylation using a spectrophotometer. GNR zeta potential was measured by dynamic light scattering (DLS).

Mouse sarcoma S-180 cells were propagated by intraperitoneal injection (5×10⁶ S-180 cells in 1 ml phosphate buffered saline (PBS)) in female CD-1 mice (4-6 weeks old) and allowed to grow until 15% weight gain was observed. Animals were then euthanized by CO₂ gas inhalation and the cells were harvested from the abdominal cavity. The cells were then washed to remove blood, diluted and subcutaneously injected into each flank of the animal (2×10⁶ cells/flank in 200 μl PBS) while anesthetized with isofluorane. Tumors were then allowed to grow until average tumor volume reached 50-100 mm³ (usually 7-10 days).

The animals were separated randomly into groups. Half received 200 μl of GNRs (9.6 mg/kg, OD=120) and the other half saline by intravenous injection through the tail vein. After 24 hours, enough time for the GNRs to accumulate in the tumor at 1.22% injected dosed based on previous experiments and other reports in the literature (Dickerson et al., 2008), the animals were anesthetized and the areas around the tumors were shaved and swabbed with 50% propylene glycol to enhance laser penetration depth. After 20 minutes EBD (10 mg/kg in 200 μl saline) was injected intravenously and a 33 gauge needle thermocouple (Omega #HYP0-33-1-T-G-60-SMPW-M) was inserted into the center of the tumor to monitor tumor temperatures. After roughly 10 seconds that temperature data was collected, an 808 nm fiber coupled laser diode (Oclaro #BMU6-808-02-R01) with collimating lens (Thorlabs #F810SMA-780, spot size=7 mm) was directed over the right tumor and radiated. Two different laser powers were used in this study (1.6 and 1.2 W/cm²) such that one group received severe and the other moderate tumor hyperthermia. After 10 minutes of radiation, the laser was turned off and tumors were allowed to cool for two minutes before removal of the temperature probe. The left tumor did not receive laser treatment to serve as an internal control.

After the animals were allowed to rest for 5 hours, enough time for the EBD to be cleared from the blood, the animals were sacrificed by CO₂ inhalation. Both tumors were collected, weighed and the EBD was extracted in 1.5 ml of formamide for 48 hrs at 60° C. The EBD content was then measured spectrophotometrically at 620 nm and divided by the weight of the tumor. The extravasation of EBD was then calculated as a ratio of the right (treated) to left (untreated) tumor and expressed as a thermal enhancement ratio (TER).

### Results and Discussion

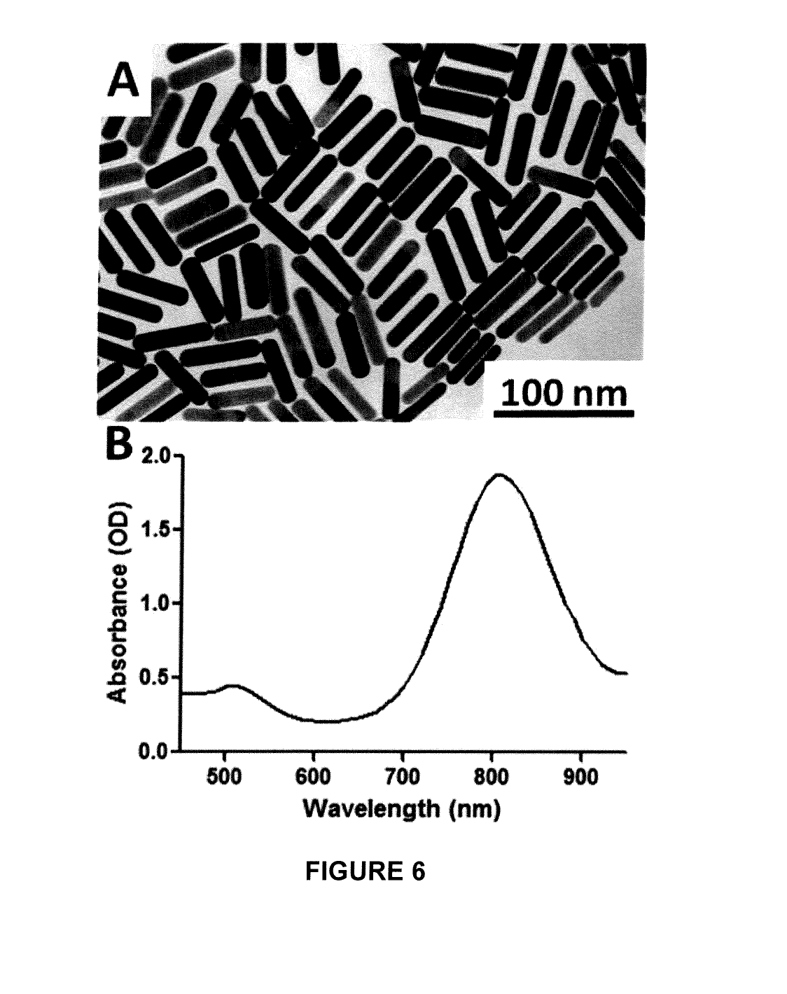

Resulting GNRs were formed with an SPR peak between 800-810 nm corresponding to a size of 60×15±6.5×2.0 nm and an aspect ratio of 4.0 (FIG. 6A). This SPR peak was easily tunable by varying silver nitrate and seed solution content (FIG. 6B).

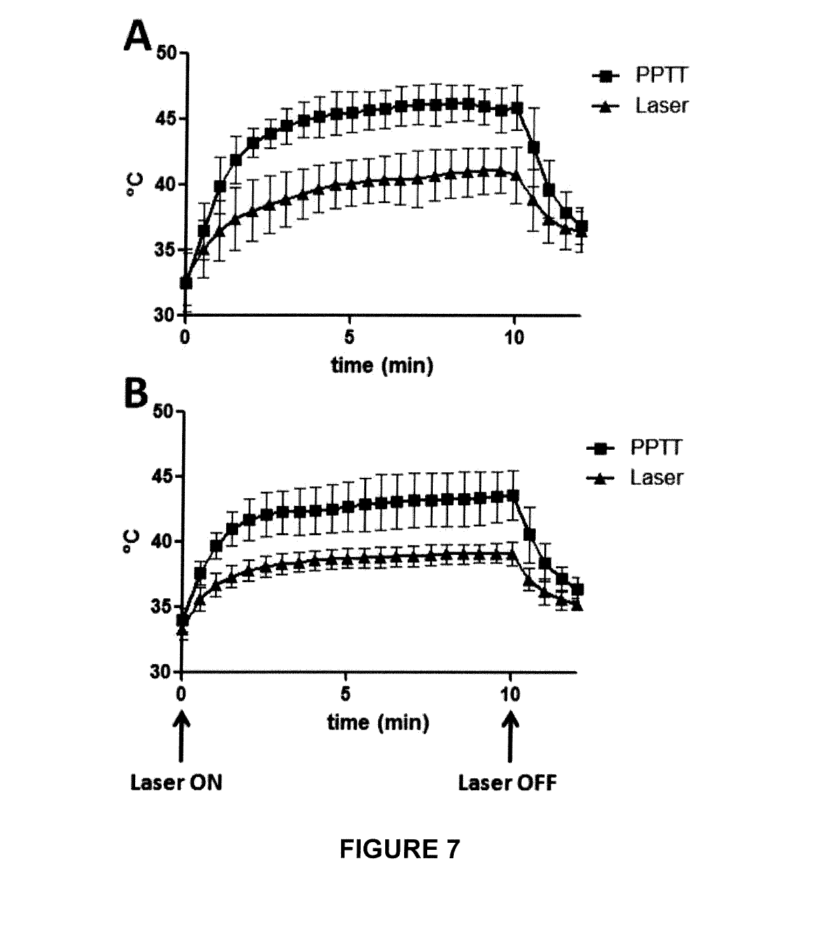

The injection of PEGylated GNRs in mice was well tolerated and no signs of distress or toxicity were observed in this and other experiments Immediately after initiation of laser treatment, temperatures inside the tumor climb rapidly and reach equilibrium within a few minutes (FIG. 7). Though treatment with laser alone (absence of GNRs) does result in some tissue heating, the presence of GNRs significantly amplified the degree of heat generation at both laser powers tested. The temperatures inside the tumors in the last 10 seconds of laser treatment were averaged and the changes in temperatures as well as final temperatures are listed in Table 1. When groups were treated with PPTT using a laser power equal to 1.6 W/cm² and 1.2 W/cm², the average equilibrium temperature inside the tumors reached 46.3° C. and 43.6° C., respectively. Therefore, by changing the laser power alone, severe and moderate hyperthermia was achieved.

After animal sacrifice 5 hours post laser treatment, the tumors were dissected out. In the animals receiving PPTT at 1.6 W/cm² significant bleeding was observed in most tumors due to conditions of severe hyperthermia. Additionally, the areas around the tumor were deeply colored in EBD indicating that the heat generated in the tumors caused the surrounding tissue to also heat. Though definitive conclusions cannot be made as to why this heating of normal tissue resulted in increased delivery of EBD, it is probable that the vessels dilated in response to insult and therefore the resulting increase in blood perfusion aided the delivery of EBD. In all other experimental groups, including animals treated with PPTT at 1.2 W/cm², no obvious hemorrhaging and local discoloration of surrounding tissue was observed.

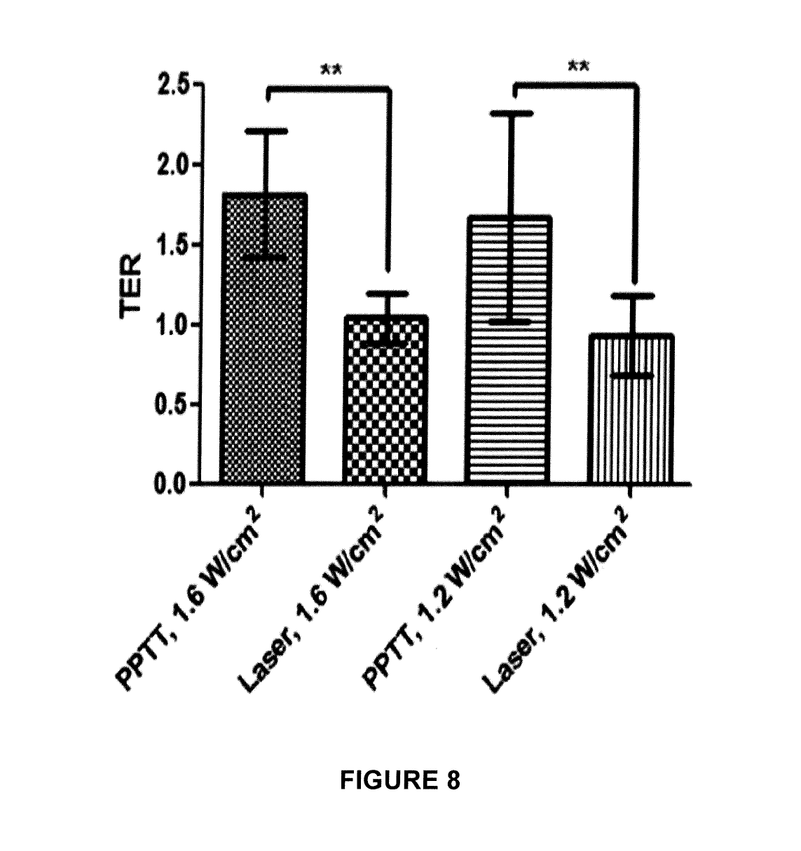

Quantification of EBD in treated and untreated tumors, expressed as a ratio, indicates that PPTT does in fact enhance the delivery of macromolecules (Table 2 and FIG. 8). When the average tumor temperature during PPTT was 46.3 and 43.6° C., the extravasation of EBD was enhanced 1.82 and 1.68-fold respectively. Though the TER is statistically different between groups with and without GNRs (p<0.01), no statistical difference is observed between both groups that received PPTT at different laser intensities. As expected, when laser treatment was applied without the presence of GNRs, the TER was around 1.0 indicating that the heat generated by laser alone, used under these study conditions, did not increase tumor microvascular permeability.

TABLE 2
Thermal Enhancement Ratio (TER)

| Group | ΔT (° C.) | Max T (° C.) | TER |
|---|---|---|---|
| ^{a}PPTT, 1.6 W/cm² | 13.7 ± 2.9 | 46.3 ± 1.3 | 1.82 ± 0.40 |
| ^{b}Laser, 1.6 W/cm² | 8.3 ± 1.8 | 41.2 ± 1.7 | 1.05 ± 0.15 |
| ^{b}PPTT, 1.2 W/cm² | 9.6 ± 2.3 | 43.6 ± 1.9 | 1.68 ± 0.65 |
| ^{c}Laser, 1.2 W/cm² | 6.0 ± 1.1 | 39.3 ± 0.8 | 0.94 ± 0.25 |

Numbers expressed as: mean ± standard deviation
^{a}N = 7
^{b}N = 6
^{c}N = 10

### PPTT Mediated GNR Induced Hyperthermia Enhances Delivery of HPMA Copolymer Conjugates in Solid Tumors

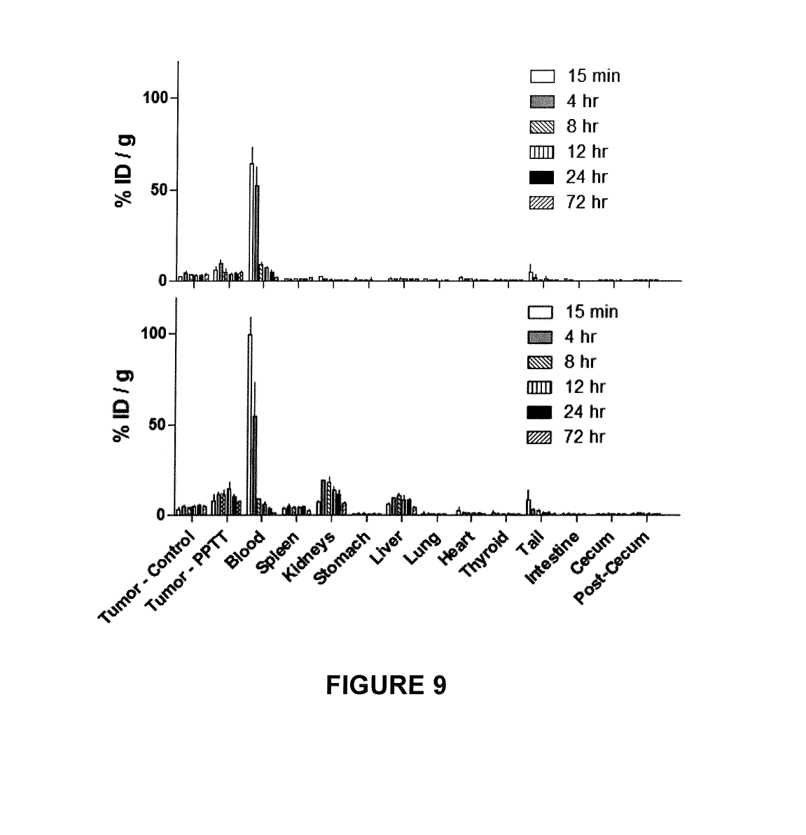

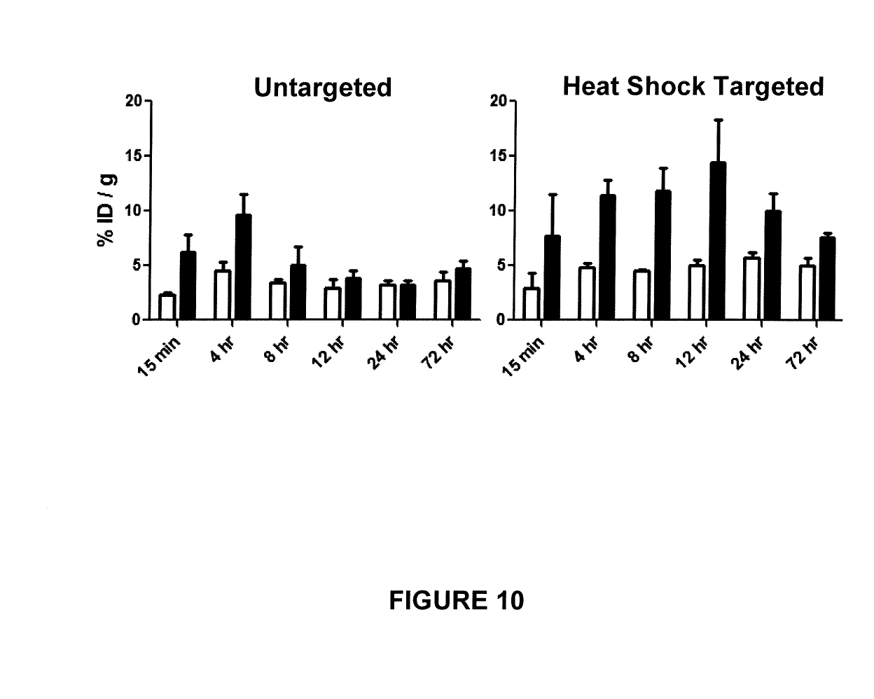

HPMA copolymers were synthesized to be 70 kDa and radiolabeled with ¹²⁵I to track their biodistribution. Untargeted copolymers with heat shock targeting copolymers containing the WIFPWIQL peptide. PEGylated GNRs synthesized above without a targeting group were first injected and allowed to accumulate for 48 hrs. Next the animals received either the radiolabled targeted or the untargeted copolymer followed immediately by 10 minutes of laser radiation (right tumor only). Animals were then sacrificed at 15 min, 4 hrs and 24 hrs and blood, tumors and organs were collected and gamma counted for radioactivity. Results indicate that both copolymers had long blood circulation and that much of the copolymer was renally excreted (FIG. 9). While the untargeted copolymer had little nonspecific organ accumulation, the targeted copolymer had some uptake by the spleen, kidneys and liver. Comparison of tumor accumulation shows that both systems had significantly more tumor localization due to PPTT compared to the tumors that were left untreated (2-3 fold increase in tumor accumulation due to PPTT) at 15 minutes and 4 hrs (FIG. 10). While after 24 hrs the untargeted copolymers had diffused back out of the tumor to the same level as the tumors left untreated, the heat shock targeted copolymers were retained. This is likely due to the fact that upon hyperthermia the GRP78 cell surface expression was upregulated and therefore enabled more copolymer binding and uptake. These results clearly demonstrate that thermal enhancement using PPTT increased delivery of HPMA copolymers and that this increased delivery is further sustained for the targeted systems.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein were apparent from consideration of the specification and practice of the compounds, compositions and methods disclosed herein. It is intended that the specification and examples be considered as exemplary.

What is claimed:

1. A method for delivering an anti-cancer agent to a tumor in a subject, the method comprising
(a) administering to the subject (i) gold particles and (ii) at least one-anti-cancer agent directly or indirectly bonded to the macromolecule and/or unbound to the macromolecule; and
(b) exposing the tumor to light for a sufficient time and wavelength in order for the gold particles to achieve surface plasmon resonance and heating the tumor.

2. The method of claim 1, wherein the gold particles are spherical particles, cages, discs or rods.

3. The method of claim 1, wherein the gold particle is a rod having a diameter from 5 nm to 100 nm and length from 10 nm to 800 nm.

4. The method of claim 1, wherein the gold particles are surface modified and have the formula I 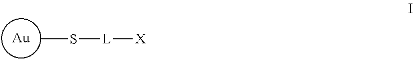
wherein
Au is a gold particle;
L is a linker; and
X is a functional group or a targeting group

5. The method of claim 4, wherein the linker is a hydrophobic linker.

6. The method of claim 4, wherein the linker comprises a hydrophilic linker.

7. The method of claim 7, wherein the hydrophilic linker comprises the polymerization product of hydroxyalkyl methacrylate (HEMA), hydroxyalkyl acrylate, N-vinyl pyrrolidone, N-methyl-3-methylidene-pyrrolidone, allyl alcohol, N-vinyl alkylamide, N-vinyl-N-alkylamide, acrylamides, methacrylamide, (lower alkyl)acrylamides and methacrylamides, hydroxyl-substituted (lower alkyl)acrylamides and methacrylamides, and any combination thereof.

8. The method of claim 7, wherein the hydrophilic linker comprises a polymer of ethylene glycol, propylene glycol, or block co-polymers thereof.

9. The method of claim 7, wherein the hydrophilic linker comprises poly(ethylene glycol) having a molecular weight from 100 to 30,000.

10. The method of claim 4, wherein the functional group is a hydroxyl group, an alkoxy group, a carboxy group, a carbonyl group, an amine group, or an amide group, an azide group, an imine group, a thiol group, a sulfonyl group, a thionyl group, a sulfonamide group, an isocyanate group, thiocyanate group, an epoxy group, a phosphate group, a silicate, or a borate group.

11. The method of claim 4, wherein the targeting group is an antibody, an antibody fragment, a saccharide, or an epitope binding peptide, or an aptamer.

12. The method of claim 4, wherein the targeting group is RGD or WIFPWIQL.

13. The method of claim 4, wherein the surface modified gold particle has the structure IV 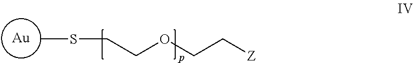
wherein
p is from 1 to 200,000; and
Z is a functional group.

14. The method of claim 13, wherein Z is a hydroxyl, an alkoxy group, a carboxy group, a carbonyl group, an amine group, or an amide group, an azide group, an imine group, a thiol group, a sulfonyl group, a thionyl group, a sulfonamide group, an isocyanate group, thiocyanate group, an epoxy group, a phosphate group, a silicate, a borate group.

15. The method of claim 13, wherein Z is alkoxy and p is from 20 to 2,000.

16. The method of claim 13, wherein Z is methoxy.

17. The method of claim 1, wherein the anti-cancer agent comprises abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gem tuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, or an analog thereof.

18. The method of claim 1, wherein the anti-cancer agent is a high Z element selected from the group consisting of iodine, lutenium, hafnium, tantalum, tungsten, rhenium, osmium, iridium, platinum, gold, thallium, lead, bismuth, radon, franceium, or any combination thereof.

19. The method of claim 1, wherein the macromolecule comprises two or more different anti-cancer agents bonded to the macromolecule.

20. The method of claim 1, wherein the macromolecule comprises dextran, dextrin, hyaluronic acid, chitosan, polylactic/glycolic acid (PLGA), poly lactic acid (PLA), polyglutamic acid (PGA), polymalic acid, polyaspertamides, poly(ethylene glycol) (PEG), poly-N-(2-hydroxypropyl)methacrylamide (HPMA), poly(vinylpyrrolidone), poly(ethyleneimine), poly(amido amine) (linear), and dendrimers comprising poly(amido amine), poly(propyleneimine), polyether, polylysine, or any combination thereof.

21. The method of claim 1, wherein the macromolecule comprises a homopolymer or copolymer prepared from a monomer comprising acrylamide, methacrylamide, N-(2-hydroxypropyl)methacrylamide, N-(2-hydroxypropyl)acrylamide, or any combination thereof.

22. The method of claim 1, wherein the macromolecule comprises a targeting group comprising monoclonal antibodies, peptides, somatostatin analogs, folic acid derivatives, lectins, polyanionic polysaccharides, or any combination thereof.

23. The method of claim 1, wherein the macromolecule comprises a targeting group, wherein the targeting group is RGD or WIFPWIQL.

24. The method of claim 1, wherein the macromolecule comprises a copolymer prepared from N-(2-hydroxypropyl)methacrylamide, geldanamycin indirectly bonded to the macromolecule by an oligonucleotide, and a targeting group having the sequence WIFPWIQL.

25. The method of claim 1, wherein the tumor comprises a breast tumor, a testicular tumor, an ovarian tumor, a lymphoma, leukemia, a solid tissue carcinoma, a squamous cell carcinoma, an adenocarcinoma, a sarcoma, a glioma, a blastoma, a neuroblastoma, a plasmacytoma, a histiocytoma, an adenoma, a hypoxic tumor, a myeloma, a metastatic cancer, bladder cancer, brain cancer, nervous system cancer, head and neck cancer, squamous cell carcinoma of head and neck, kidney cancer, lung cancers including small cell lung cancer and non-small cell lung cancer, neuroblastoma/glioblastoma, ovarian caner, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer, colorectal cancers, prostatic cancer, or pancreatic cancer.

26. The method of claim 1, wherein the gold particles are administered first followed by the administration of the macromolecule.

27. The method of claim 1, wherein the macromolecule is administered first followed by the administration of the gold particles.

28. The method of claim 1, wherein the gold particles and the macromolecule are administered simultaneously.

29. The method of claim 1, wherein the gold particles and the macromolecule are administered intraveneously.

30. The method of claim 1, wherein the tumor is exposed to light produced from a laser diode light source and heated.

31. The method of claim 1, wherein the tumor is exposed to light produced from a laser diode light source comprising a dose from 0.25 to 4 W/cm² for a duration of 1 to 60 minutes.

32. The method of claim 1, wherein the method reduces or prevents tumor cell proliferation.

33. The method of claim 1, wherein the method kills tumor cells.
